(19) United States
(12) Patent Application Publication
(10) Pub. No.: US 2026/0356675 A1
(43) Pub. Date: Nov. 12, 2026

(54) CLEANING OF CO2-CONTAINING FEED GASES

(71) Applicant: TOPSOE A/S, Kgs. Lyngby (DK)

(72) Inventors: Martin ØSTBERG, Kgs. Lyngby (DK); Thomas Sandahl CHRISTENSEN, Kgs. Lyngby (DK)

(73) Assignee: TOPSOE A/S, Kgs. Lyngby (DK)

(21) Appl. No.: 19/486,576

(22) PCT Filed: May 23, 2024
(86) PCT No.: PCT/EP2024/064176

§ 371 Date: Nov. 21, 2025

(30) Foreign Application Priority Data
May 24, 2023 (DK) PA202330042

(51) Int. Cl. C01B 32/50; B01D 53/86; C07C 4/06

(52) U.S. Cl. CPC C01B 32/50; B01D 53/864; C07C 4/06; B01D 2256/22; B01D 2257/30; B01D 2257/7022

(57) ABSTRACT

A process is provided for cleaning a CO₂-rich gas stream from one or more impurities selected from sulfur-containing compounds, higher hydrocarbons and aromatic hydrocarbons, The process comprises the step of passing the CO₂-rich gas stream together with a steam feed over a guard material, comprising a catalyst active in the conversion of higher hydrocarbons into methane in the presence of said steam feed (i.e. a pre-reforming catalyst).

### TEHCNICAL FIELD

The present invention relates to a process for cleaning a CO₂-rich gas stream, in particular for removing impurities such as hydrocarbons, and remaining sulfur-containing impurities after the hydro-desulfidation (HDS) section.

### BACKGROUND

Carbon dioxide is commercially available in different grades. Typically, “food grade” or “beverage grade” CO₂ has a purity of 99.9%. However, for processes involving catalytic conversion of CO₂ to other chemical products (e.g., power-to-X), impurities such as sulfur-containing compounds and oxygen in the CO₂ stream may poison the synthesis catalyst, even when present at concentrations of 50-100 ppbV (0.000005-0.00001%). Contents of hydrocarbons especially C₂+ may also lead to undesired reactions on the CO₂ conversion catalysts together with content of nitrogen species.

Despite the high purity of certain CO₂ sources, it has been discovered that further purification is required to avoid catalyst poisoning of downstream synthesis catalyst. It would also be advantageous if multiple impurities of different types (e.g. sulfur-containing compounds, higher hydrocarbons and aromatic hydrocarbons) could be removed in a single process without addition of oxygen or air that will lead to a loss of hydrogen downstream.

Systems and processes for purification of CO₂ streams are known from e.g. EP2457636, CN112999843 and CN112957872.

### SUMMARY

It has been found by the present inventor(s) that introduction of a catalytic guard after an HDS section can protect the downstream synthesis/CO₂ conversion catalyst from impurities and undesired side reaction that may limit the lifetime of the catalyst. The guard is a nickel-based catalyst, noble metal or combination of these with ability to convert content of hydrocarbons such as paraffines, aromatics and oxygenates contained in the CO₂ stream after a HDS section after mixing with a stream of steam. Nitrogen species other than molecular nitrogen, such as nitrogen oxides and ammonia, will also react, and the catalytic guard will adsorb sulfur on the nickel surface reducing active sites on the guard, but preventing an activity loss and reduced lifetime of the catalyst downstream the guard.

It has been found by the present inventor(s) that a prereformer catalyst may be used as a guard for CO₂ feeds. This will ensure to capture sulfur slip from upstream sulfur cleaning and it will enable removal of hydrocarbons and convert these to methane.

So, in a first aspect the present invention relates to a A process for cleaning a CO₂-rich gas stream, said CO₂-rich gas stream comprising at least 80 wt % CO₂ and one or more impurities selected from:
sulfur-containing compounds;
higher hydrocarbons; and
aromatic hydrocarbons,
nitrogen species,
   wherein said process comprises the step of:
passing the CO₂-rich gas stream together with a steam feed over a guard material, comprising a catalyst active in the conversion of higher hydrocarbons into methane in the presence of said steam feed, and adsorbing one or more of said impurities on said guard material, to provide a cleaned CO₂-rich gas stream.

This provides an extra sulfur guard to handle possible content of hydrocarbons in low concentrations (ppm level) in the CO₂ feed.

The present invention also relates to a process for the production of chemical or fuel stream, said process comprising cleaning a CO₂-rich gas stream in a process as described herein, followed by feeding the cleaned CO₂-rich gas stream to a synthesis section, optionally in admixture with a hydrogen feed, and outputting a chemical or fuel stream from said synthesis section. Chemicals and fuels produced by such a process include but are not limited to H₂, CO, MeOH, formaldehyde, DME, FT-based fuels, gasoline, synthetic aviation fuels.

Additional aspects are presented in the following description text, figures and claims.

### LEGENDS

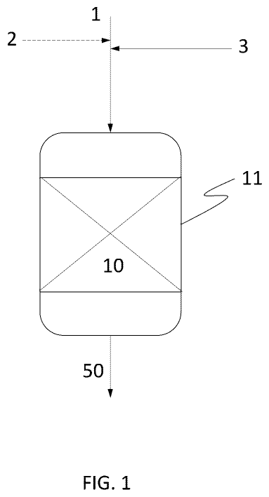

FIG. 1 shows a simple layout of one embodiment of the process of the invention.

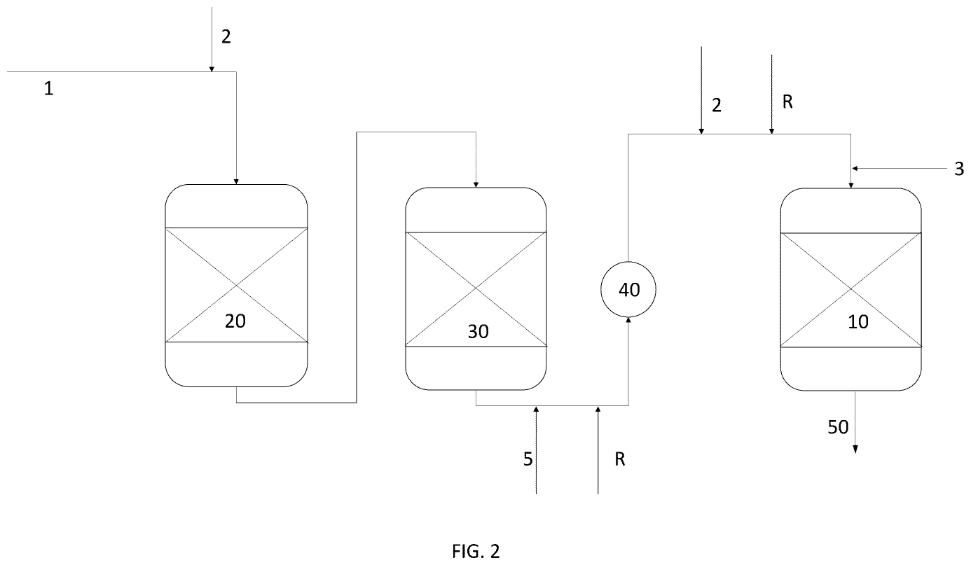

FIG. 2 shows a more advanced layout of an embodiment of the process of the invention

### DETAILED DISCLOSURE

Unless otherwise specified, any given percentages for gas content are % by volume. All feeds are preheated as required. Unless specified, the concentrations will be given on dry basis, i.e. without taking any water present into account.

Higher hydrocarbons are all hydrocarbons containing more than one carbon atom in the molecule meaning practically all hydrocarbons except methane.

Syngas is used as reference for a synthesis gas, a gas mixture comprising hydrogen, carbon monoxide, carbon dioxide and typically water as steam and methane. It is referred to as syngas/synthesis gas because it is the feed for a downstream catalytic synthesis leading to the desired product. In some application the feed downstream the referred purification can be mixed with hydrogen and be used as synthesis gas e.g. for methanol synthesis in other applications, the purified gas may after mixing with hydrogen and optionally steam need conversion in a reverse water gas shift reactor (RWGS) or combined RWGS and methanation reactor to form the final synthesis gas for the synthesis of the final product.

A cleaned CO₂ stream is defined as the outlet stream from the CO₂ cleaning process, in which minimum 95% of the combined sulfur containing impurities in the feed is removed or the sum of sulfur containing impurities in the clean CO₂ stream is lower than 50 ppbV (parts per billion by volume), preferably lower than 1 ppbV.

The sum of sulfur containing impurities should be understood as sulfur equivalents, i.e. 10 ppbV SO₂ correspond to 10 ppbV sulfur and 10 ppbV CS₂ correspond to 20 ppbV sulfur

The proposed CO₂ cleaning solution ensures that the feed gases for any downstream conversion to synthesis gas and synthesis for chemicals like MeOH, (methanol), DME, (dimethyl ether), FT (Fischer Tropsch)), synthetic fuels etc. will be unproblematic with regard to by-products from higher hydrocarbons, and can be made without poisoning from sulfur of the downstream synthesis catalyst by sulfur. This will ensure that operation can be made over time and allow catalyst lifetime as expected for industrial catalyst.

In a first aspect, therefore, a process for cleaning a CO₂-rich gas stream is provided. Suitably, the CO₂-rich gas stream to the process has been treated in a HDS section removing most of the sulfur and reacting possible content of oxygen.

The CO₂-rich gas stream provided to the process comprises at least 80 wt %, such at least 90 wt % CO₂, such as at least 95 wt % CO₂, such as at least 99.0 wt % CO₂, preferably at least 99.5 wt % CO₂, more preferably as at least 99.9 wt % CO₂. The CO₂-rich gas stream is thus already of high purity prior to the process of the present invention.

Suitably, the CO₂-rich gas stream is derived from a renewable source, such as:
combustion or gasification of a lignocellulosic biomass such as wood products, algae, grass, forestry waste and/or agricultural residue;
combustion or gasification of municipal waste, in particular the organic portion thereof, where the municipal waste is defined as a feedstock containing materials of items discarded by the public, such as mixed municipal waste given in EU Directive 2018/2001 (RED II), Annex IX, part A;
microbial conversion of nitrogen-rich renewable feedstock such as manure or sewage sludge;
fermentation of hydrocarbon (sugar) rich feed streams such as corn, sugar cane and beets;
a CO₂ recovery unit from chemical production e.g. a hydrogen or an ammonia production plant, where CO₂ is removed from the product gas.

The CO₂-rich gas stream can also be obtained from direct air capture processes, metallurgical processes, cement production or fossil fuel combustion.

The CO₂ concentration in some of the above-mentioned gas streams may typically be too low for further chemical processing and a concentration step may be required to increase the CO₂ concentration to the desired value as mentioned above.

The CO₂-rich gas stream comprises one or more impurities selected from sulfur-containing compounds, higher hydrocarbons, aromatic hydrocarbons and nitrogen species.

The CO₂ rich gas is mixed with a stream of steam and led to the catalytic guard reactor. The amount of steam added to the CO₂ rich gas is dependent on the content of hydrocarbons that should be converted and to the content of hydrogen in the CO₂ rich gas after the HDS section. The total molar amount of steam added should not be higher than 100 times the molar flow of hydrogen coming with the CO₂ rich gas preferably not higher than 40 times tbe molar flow of hydrogen. If this amount of steam is not sufficient to ensure a conversion of the higher hydrocarbons additional hydrogen should be added to the CO₂ rich gas. The amount of higher hydrocabons may be increased by addition of a recycle stream coming from separation of the downstream obtained product gas after the catalytic synthesis recycling a light end of the obtained product gas. The composition of this recycle stream depends on the downstream synthesis.

The amount of hydrogen added is however limited by a desire not to obtain a significant temperature rise due to the exothermal reaction by methanation that occurs when CO₂ rich gas mixed with substantial amounts of hydrogen is reacted over a nickel or noble metal catalyst. Actually, the temperature out of the catalytic guard reactor may be controlled by the addition of additional hydrogen to the CO₂ rich gas.

The CO₂ rich gas with steam—and possibly hydrogen—is passed over a guard material, comprising a catalyst active in the conversion of higher hydrocarbons into methane in the presence of said steam feed. A “catalyst active in the conversion of higher hydrocarbons into methane” is typically a pre-reforming catalyst.

This catalyst may be a nickel-based catalyst, a noble metal based catalyst or a combined nickel and noble metal catalyst. This can be catalyst having carrier materials of alumina, calcium aluminate, magnesium aluminate, promoted-alumina or spinels, where promotion metals could be Ti, La, Ce, Zr, or Y. The content of nickel could be 15-60 wt % preferably 25-55 wt %. Content of noble metal will be lower 0.5-10 wt % and when combined with nickel at levels of 0.1-2 wt %. The catalyst may be an impregnated catalyst or a co-precipitated catalyst. It may be pre-reduced prior to installation, or it can be reduced in the reactor for activation depending on the requirements for reduction to form the active metallic nickel or noble metal phase from the respective oxides.

The catalyst will have the ability to convert higher hydrocarbons to methane, carbon monoxide, hydrogen and water through the following reactions also involving the CO₂. First the reforming reaction:

CₓH_{y}+x H₂O=>x CO+(y/2+x) H₂

This will be followed by methanation:

CO+3 H₂ó CH₄+H₂O

The high amount of CO₂ will also lead to reverse water gas shift as equilibrating the concentrations of CO₂, CO, H₂ and H₂O:

CO₂+H₂ ó CO+H₂O

These products are all normal constituents in a synthesis gas except for methane.

Another feature of the nickel catalyst is the ability to adsorb sulfur on the nickel surface. At low temperature (below 500 °C) practically all sulfur will be adsorbed preventing sulfur slip to the downstream catalyst/catalysts. The adsorbed sulfur removes catalytic activity for the above catalytic reactions and it is therefore necessary to ensure that both adsorption capacity and catalytic activity are sufficient for the catalytic guard during its lifetime.

The catalyst will also have the ability to convert nitrogen species such as nitrogen oxides and ammonia. The nitrogen oxides will be reduced to ammonia, and the ammonia will reach an equilibrium according to the following catalytic reaction:

2 NH₃ ó N₂+3 H₂

The ammonia level leaving the catalytic guard reactor will depend on the nitrogen content in the CO₂ rich gas, the hydrogen concentration, the pressure and the temperature. This ammonia decomposing reaction leads to formation of molecular nitrogen and hydrogen and equilibria is directed towards the products with increasing temperature and lower pressure, why a low ammonia slip can be obtain having a low nitrogen (N₂) concentration in the CO₂ rich gas and having the guard at high temperature and low pressure relevant if the downstream catalysts are sensitive to ammonia.

The guard reactor will be adiabatic having inlet temperatures between 250-500 °C. Care should be taken that the reactor does not have significant temperature differences, especially temperature rises. For this reason, the amount of hydrogen added should be limited to prevent significant methanation taking place.

Suitably, the CO₂-rich gas stream comprises one or more impurities selected from from H₂S, DMS (dimethyl sulfide) and COS. The total content of SO₂ in the CO₂-rich gas stream e.g. at the inlet of said reactor vessel is 0.1-100 ppm SO₂, such as 1-50 ppm SO₂.

Generally, therefore, the process comprises the step of: passing the CO₂-rich gas stream together with a steam feed over a guard material, comprising a catalyst active in the conversion of higher hydrocarbons into methane in the presence of said steam feed (i.e. a prereforming catalyst), and adsorbing one or more of said impurities on said guard material, to provide a cleaned CO₂-rich gas stream.

The steam feed is typically a high-purity steam feed, i.e. it comprises at least 99 wt % H₂O, such as at least 99.5 wt % H₂O.

The operating pressure of the catalyst and adsorption system will be in the range 1 to 90 barg, depending on the feed pressure of the CO₂ and other feed gases and the pressure of the downstream synthesis plant. Generally, a lower pressure will be more beneficial with regard to minimizing the formation of undesired side products, but it comes with a cost of larger equipment.

In one aspect of the invention, the CO₂-rich gas stream is passed over the guard material, together with (e.g. in admixture with) a first hydrogen-rich feed to ensure sufficient hydrogen for the conversion of higher hydrocarbons.

The higher hydrocarbons removed in the process according to the invention may be selected from C2-C6 alkanes, C2-C6 oxygenates (i.e. hydrocarbons containing one or more oxygen atoms), and combinations thereof.

The higher aromatic hydrocarbons may be selected from C6-C8 aromatic hydrocarbons, C9-C12 aromatics hydrocarbons, and combinations thereof.

In the process, the CO₂-rich gas stream may be subjected to one or more desulfurisation steps, preferably two or more desulfurisation steps prior to being passed over the guard material. Desulfurisation may take place via a process described in co-pending DK patent applications PA202300339 and PA202300340, which are hereby incorporated by reference.

According to one aspect, desulfurisation can be a two-step process. According to this aspect, the process comprises:
Passing the CO₂-rich gas stream over a first sulfur guard material, followed by
passing the CO₂-rich gas stream over a second sulfur guard material and adsorbing one or more sulfur-containing compounds on said first and said second sulfur guard materials,
prior to the CO₂-rich gas stream being passed over said guard material.

Preferably, the first sulfur guard material is characterized by being prone to H₂S slip, while the second sulfur guard material, is characterized by not exhibiting H₂S slip. Optionally, water may be removed from the CO₂-rich gas stream between first and second guard materials.

The first and second sulfur guard materials can have the same composition, wherein the second sulfur guard material is at different process conditions which allow for no H₂S slip operation. That could be accomplished by installing a water removal unit between the first and second sulfur guard material and/or lowering the temperature between the first and second sulfur guard material by installing a heat exchange unit.

The second sulfur guard material can have a chemical composition different from the first sulfur guard material, e.g. a ZnO based first guard material and a Cu—Zn—Al based second sulfur guard material. Even so it may be beneficial to operate the two guard materials at different process conditions as suggested above.

In one aspect, the CO₂-rich gas stream is subjected to a pre-heating step, prior to being passed over said guard material.

In one aspect, the cleaned CO₂-rich gas stream comprises less than 50 ppbV, preferably less than 10 ppbV and most preferably less than 5 ppbV sulfur. In another aspect, the cleaned CO₂-rich gas stream comprises less than 100 ppm, preferably less than 50 ppm, more preferably less than 10 ppm higher hydrocarbons. In yet another aspect, the cleaned CO₂-rich gas stream comprises less than 10 ppm, preferably less than 5 ppm and most preferably less than 1 ppm aromatic hydrocarbons. In a further aspect, the cleaned CO₂-rich gas stream comprises less than 10 ppm, preferably less than 1 ppm and most preferably less than 0.1 ppm alcohols.

In the process described herein, the step of passing the CO₂-rich gas stream together with a steam feed over a guard material, may be carried out at a temperature of between 250 and 550 °C, preferably between 300 and 400 °C. Operation pressure can vary dependent on optimal design of the overall process from 5 barg to 80 barg.

The amount of catalyst may also vary, having typically a feed flow of CO₂ rich gas and steam between 1,000 Nm³/h/m³ catalyst to 10,000 Nm³/h/m³ catalyst.

A process is also provided for the production of a chemical or fuel stream, said process comprising cleaning a CO₂-rich gas stream in a process as described herein, followed by feeding the cleaned CO₂-rich gas stream to a synthesis section, optionally in admixture with a hydrogen feed, and outputting a chemical or fuel stream from said synthesis section.

### Specific Embodiments of the Invention

FIG. 1: The CO2 rich gas stream (1) (e.g. from a HDS section) may optionally be mixed with a hydrogen stream (2) before mixing with a steam stream (3). The CO2-rich gas stream is fed to the catalytic guard reactor (11) comprising guard material (10). The cleaned CO₂-rich gas stream (50) leaving the catalytic guard reactor (11) will be sent for downstream conversion.

FIG. 2: The CO₂ rich gas (1) is mixed with a hydrogen stream (2) and let through a hydrodesulfurization section consisting of a first reactor (20) containing a hydrogenation catalyst and subsequently of a second reactor (30) containing a sulfur adsorption or a sulfur adsorption/chemisorption guard. The hydro desulfurized CO₂ rich gas can then optionally be mixed with a second hydrogen stream (5) and/or optionally a recycle stream (R) recycling unconverting synthesis gas or light-end hydrocarbons from a downstream synthesis. The recycle stream (R) will typically be a mixture of lighter hydrocarbons, CO, CO₂ and H₂.

The CO₂ rich gas mixed with hydrogen and steam or only with steam (3) may be preheated in a preheater (10) to the desired inlet temperature. A heater (40) can be used to heat the CO₂ rich gas either before or after mixing with the second hydrogen stream (5) or (2) and/or the recycle stream (recycle). Before entering the catalytic guard reactor (50) a stream of steam (3) is mixed with the stream. The cleaned CO₂ rich gas (50) leaves the chemical guard reactor (10) and is sent to the downstream catalytic processes. The optionally second hydrogen stream (5) or (2) will be the hydrogen needed for the production of the synthesis gas for the downstream catalytic process. This hydrogen stream may optionally also be mixed into the cleaned CO2 rich gas (50) after the catalytic guard reactor (10).

The present invention has been described with reference to a number of aspects and figures. However, the skilled person is able to select and combine various aspects within the scope of the invention, which is defined by the appended claims. All documents referenced herein are incorporated by reference.

1. A process for cleaning a CO₂-rich gas stream, said CO₂-rich gas stream comprising at least 80 wt % CO₂ and one or more impurities selected from:
sulfur-containing compounds;
higher hydrocarbons;
aromatic hydrocarbons; and
nitrogen species,
wherein said process comprises the step of:
passing the CO₂-rich gas stream together with a steam feed over a guard material, comprising a catalyst active in the conversion of higher hydrocarbons into methane in the presence of said steam feed, and adsorbing one or more of said impurities on said guard material, to provide a cleaned CO₂-rich gas stream.

2. The process according to claim 1, wherein the CO₂-rich gas stream comprises at least 90 wt % CO₂.

3. The process according to claim 1, wherein the CO₂-rich gas stream is passed over the guard material together with a first hydrogen-rich feed.

4. The process according to claim 1, wherein the catalyst is a prereforming catalyst.

5. The process according to claim 1, wherein the sulfur-containing compounds are one or more compounds selected from COS, DMS and H₂S.

6. The process according to claim 1, wherein the higher hydrocarbons are selected from C2-C6 alkanes, C2-C6 alkenes, C2-C6 alkynes, and combinations thereof.

7. The process according to claim 1, wherein the higher aromatic hydrocarbons are selected from C6-C8 aromatic hydrocarbons, C9-C12 aromatics hydrocarbons, and combinations thereof.

8. The process according to claim 1, wherein the CO₂-rich gas stream is subjected to one or more desulfurization steps, preferably two or more desulfurization steps prior to being passed over said guard material.

9. The process according claim 1, wherein the CO₂-rich gas stream is subjected to a pre-heating step, prior to being passed over said guard material.

10. The process according to claim 1, wherein the cleaned CO₂-rich gas stream comprises less than 50 ppbV sulfur.

11. The process according to claim 1, wherein the cleaned CO₂-rich gas stream comprises less than 100 ppm higher hydrocarbons.

12. The process according to claim 1, wherein the cleaned CO₂-rich gas stream comprises less than 10 ppm aromatic hydrocarbons.

13. The process according to claim 1, wherein the cleaned CO₂-rich gas stream comprises less than 10 ppm alcohols.

14. The process according to claim 1, wherein the step of: passing the CO₂-rich gas stream together with the steam feed over a guard material, is carried out at a temperature of between 250 and 550 °C.

15. A process for the production of a chemical or fuel stream, said process comprising cleaning a CO₂-rich gas stream in a process according to claim 1, followed by feeding the cleaned CO₂-rich gas stream to a synthesis section, optionally in admixture with a hydrogen feed, and outputting a chemical or fuel stream from said synthesis section.
